(19) United States
(12) Patent Application Publication
(10) Pub. No.: US 2012/0138078 A1
(43) Pub. Date: Jun. 7, 2012

(54) COSMETIC ASSEMBLY FOR MAKING UP AND/OR CARING FOR KERATIN MATERIALS

(75) Inventor: Audrey Ricard, La Varenne (FR)

(73) Assignee: L'OREAL, Paris (FR)

(21) Appl. No.: 13/318,972

(22) PCT Filed: May 4, 2010
(86) PCT No.: PCT/IB2010/051948

§ 371 Date: Feb. 16, 2012

Related U.S. Application Data
(62) Division of application No. 61/213,826, filed on Jul. 20, 2009.

(30) Foreign Application Priority Data
May 6, 2009 (FR) 0953030

(51) Int. Cl. A61K 8/92; A61Q 1/12; A61Q 1/06; A61Q 1/08; A45D 33/26; A45D 40/26

(52) U.S. Cl. 132/200; 132/317; 132/293

(57) ABSTRACT

The present invention relates to a cosmetic assembly for making up and/or caring for keratin materials, comprising at least:—one container containing at least one solid cosmetic composition, the said composition comprising at least one liquid fatty phase, at least one wax and at least 5% by weight of pigments relative to the total weight of the said composition, and having a melting point of greater than 30° C., and—one application device for applying the composition, equipped with an application member comprising an application surface that is capable, in response to its movement in engagement with the keratin materials, of rotating about at least one axis or one centre of rotation.

The present invention is directed towards proposing a new method for making up and/or caring for keratin materials and more particularly dedicated to the application of a solid composition to a keratin material, in particular to the skin or the lips, and more particularly the skin.

In general, cosmetic compositions dedicated to caring for and/or making up the skin and more particularly the face are applied according to the following method: one or more spot deposits of the composition are made on one or more localized areas of the face, and this or these deposits are generally spread out with the fingers or a sponge to cover the entire surface of the face.

For the reasons that follow, it would be advantageous to have available a new method for applying makeup and/or care compositions of this type.

Firstly, this conventional application method cannot guarantee an applied film thickness that is uniform over all the areas of the face. Generally, the areas where the composition has been spotted on benefit from a greater thickness of makeup film. Secondly, the areas of the face that are the most remote from the abovementioned area(s) are coated with a makeup film of smaller thickness. This non-uniformity of deposition generally becomes apparent in the makeup effect. For obvious reasons, it is not appreciated by the user. It is especially incompatible with the manifestation of “natural” rendering in terms of the makeup effect.

Obtaining a uniform deposit is all the more essential in the context of makeup compositions incorporating pigments. In point of fact, it is imperative, in order to obtain good colour properties, both on the visual aspect of the product itself and on the makeup result after using it, to be able to ensure, firstly, perfect dispersion of the pigments in the makeup composition and, secondly, uniformity of deposition during application.

Moreover, this application method may more particularly, in the case of spreading with the fingers, cause a problem as regards cleanliness, or even hygiene.

Finally, users are always in search of novel application techniques that are innovative in terms of implementation, of visual effect afforded by the type of makeup deposit that they form, or even in terms of sensory sensations generated at the time of application.

The present invention is precisely directed towards favourably meeting the various expectations outlined above.

Thus, according to one of its aspects, the present invention relates to a cosmetic assembly for making up and/or caring for keratin materials, comprising at least:
one container containing at least one solid cosmetic composition, in particular in the form of a cast product, the said composition comprising at least one liquid fatty phase and at least one wax and having a melting point of greater than 30° C., and
one application device for applying the composition, equipped with an application member comprising an application surface that is capable, in response to its movement in engagement with the keratin materials, of rotating about at least one axis or centre of rotation.

According to one particularly advantageous embodiment, the present invention more specifically relates to a cosmetic assembly for making up and/or caring for keratin materials, comprising at least:
one container containing at least one solid cosmetic composition, in particular in the form of a cast product, the said composition comprising at least one liquid fatty phase, at least one wax and at least 5% by weight of pigments relative to the total weight of the said composition, and having a melting point of greater than 30° C., and
one application device for applying the composition, equipped with an application member comprising an application surface that is capable, in response to its movement in engagement with the keratin materials, of rotating about at least one axis or centre of rotation.

For the purposes of the present invention, the term “solid” means a composition of high consistency, which keeps its shape during storage. As opposed to “fluid” compositions, it does not flow under its own weight. It is advantageously characterized by a hardness as defined below.

The term “cast product” denotes a mass of product whose cohesion is ensured by solidification of at least one of its constituents during implementation. The composition may be hot-cast in a mould, and the solidification results from its cooling.

As developed hereinbelow, these products generally comprise, on the one hand, a pulverulent phase especially comprising pigments and optionally fillers, and, on the other hand, a fatty phase as binder, comprising fatty substances, which is intended to give the finished product softness and emollience properties and to promote its adhesion to the keratin material, especially the skin or the lips.

As regards the application device, it is not limited to a single variant of application member.

As emerges from the appended figures, several embodiment variants may be envisaged provided that they are compatible with a rotation of their application surface about at least one axis or centre of rotation, in response to a movement of this surface on contact with the keratin material, generally the skin, to be treated.

It is understood that this capacity to undergo rotation in response to a movement of the application surface over a keratin material is also verified during the movement of this application surface over the solid composition to be taken up for makeup purposes.

As regards the different embodiment variants proposed hereinbelow, these movements over the keratin material and over the solid composition may be manifested consecutively, as is the case for the variants illustrated in FIGS. 1 to 5, generally in chronological order at the surface of the composition and then at the surface of a keratin material, but also simultaneously as is the case for the variant illustrated in FIG. 6 in which the movement of the application surface over the keratin material initiates its movement over the solid composition, the two movements then being manifested together. The application member may be defined by a roll, a ball or a strip arranged around two rolls with parallel axes of rotation.

The application member is dedicated firstly to taking up some of the composition present in the associated container, and secondly to applying the composition thus taken up onto the surface of a keratin material.

In a first stage, the passage of the outer surface of the application member and more particularly of the roll onto the surface of the composition results in rotation of the composition. The application member is then impregnated or coated with composition by friction and/or pressure.

In a second stage, consecutive passage of the outer surface of the application member, and more particularly of the roll, at the surface of a keratin material, especially facial skin, causes it to rotate and to spread the composition that is on its outer surface.

In one embodiment variant, the application member may be equipped with a removable brake making it possible, if necessary, to block or brake its free rotation, during the uptake and/or application.

The outer face of the application member is formed, at least at the surface, from a material that is suitable for taking up the cosmetic composition and retaining it until its consecutive application to the skin.

For example, the application surface dedicated to be in contact with a keratin material may especially be a foam with open or closed cells, optionally flocked, a flocking, an elastomer, a sintered material, a woven material or a nonwoven material.

The outer surface of the application member may or may not be smooth. Thus, this application member may advantageously have reliefs at the surface, generally outward-curving and rounded, advantageous for affording a combined massage effect.

According to one preferred variant, its appearance resembles that of an applicator roll like those used for applying paint. Such applicators used for cosmetic purposes are especially described in documents FR 985 064 (lip makeup powder), FR 1 524 192 (powder), FR 1 281 338 (compact product) and FR 2 848 790 (solid or fluid cosmetic product).

Thus, the application member is advantageously in the form of a hollow or non-hollow roll, which rotates about an axis of rotation. This axis of rotation may advantageously be arranged perpendicular to the longitudinal axis of the device.

When the application member is a roll, the product may have an uptake surface of rectangular shape, having a dimension larger than the length of the roll, for example a width of between 1.01 and 1.2 times the length of the roll. The bottom of the housing containing the product may be rounded concave, so as to facilitate the uptake of the product until it is completely empty, by rolling the application member along the entire length of the housing containing the product.

The application member may or may not be integrally attached to the container containing the composition during use or storage. According to one preferred variant, it is not integrally attached.

The container may also be or not be integrally attached to the application member during the application of the product to the keratin materials.

According to one preferred embodiment, the application device and the container are combined in the same conditioning, for example the same case provided with a base containing the product and having a housing for receiving the applicator and a lid that is mobile relative to the base, for example articulated thereon.

As emerges from the examples given below, the use of a rotary member for applying a solid composition to the surface of a keratin material and more particularly of the skin makes it possible to obtain a makeup effect different from that obtained with a conventional applicator such as a sponge or a powder puff. In particular, the makeup effect obtained according to the invention proves to be significantly improved in terms of uniformity of the deposit. The film obtained may be very thin, uniform and free of traces and give a discreet or even natural makeup effect.

Such an application device makes it possible in particular to obtain, in the context of compositions incorporating pigments, good homogeneity of the colour effect of the makeup.

During application, the application member can rotate without sliding on the skin. Several successive passages may be made at the same place, depending, for example, on the desired colour intensity. During successive passages, the user can slightly modify the direction of rolling, so as to fade out the edges of the deposit of product.

Moreover, the use of an application member in accordance with the invention for depositing a solid composition at the surface of a keratin material and more particularly of the skin makes it possible to obtain a massage effect on the skin, with a sensation of well-being.

Thus, the use of the application method according to the invention proves, firstly, to be easy, and, secondly, to be of a nature to afford the user pleasant sensory sensations.

The composition according to the invention may be a makeup and/or a care composition for the skin, in particular of the face and/or the body, and may constitute a makeup rouge, an eyeshadow, a face powder, a foundation, a concealer product, a lipstick, a body makeup product, a body or face care product or an antisun product.

More especially, but not exclusively, the invention relates to a foundation composition and in particular a cast foundation.

According to another aspect, the invention relates to a process for making up and/or caring for a keratin material, in which:
an amount of a solid composition, especially in the form of a hot-cast product, is taken up using an application member comprising an application surface that is capable, in response to its movement in engagement with keratin materials, of rotating about an axis or a centre of rotation, especially as defined hereinbelow, and
the composition thus taken up is applied using the said application member onto the said keratin material, the said composition comprising at least one liquid fatty phase, at least one wax and having a melting point of greater than 30° C.

In particular, the invention relates to a process for making up and/or caring for a keratin material, in which:
an amount of a solid composition, especially in the form of a hot-cast product, is taken up using an application member comprising an application surface that is capable, in response to its movement in engagement with keratin materials, of rotating about an axis or a centre of rotation, especially as defined hereinbelow, and
the composition thus taken up is applied using the said application member onto the said keratin material, the said composition comprising at least one liquid fatty phase, at least one wax and at least 5% by weight of pigments relative to the total weight of the said composition, and having a melting point of greater than 30° C.

### EXAMPLES OF APPLICATION DEVICES AND OF COSMETIC ASSEMBLIES FOR IMPLEMENTING THE INVENTION

A few examples, among others, of application devices that may be used have been represented in the figures.

In the drawing:

FIG. 1 schematically represents, in perspective, an example of a packaging and application device according to the invention,

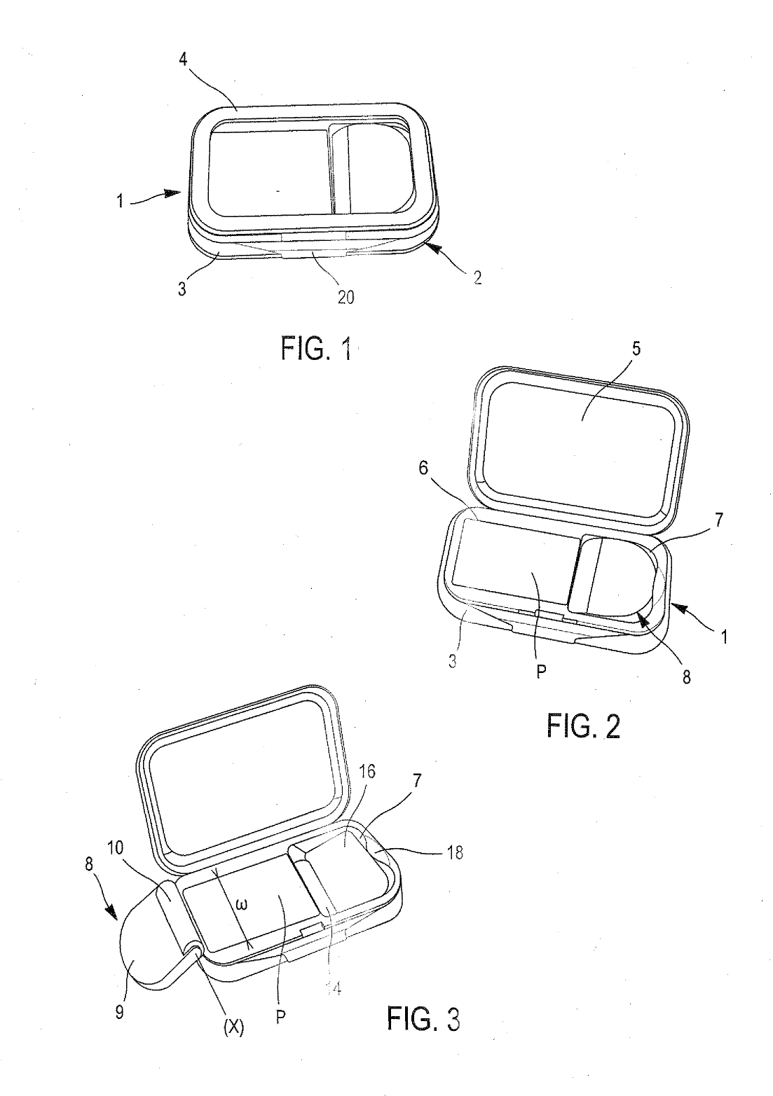

FIG. 2 represents the device of FIG. 1, with the case open,

FIG. 3 represents the device of FIG. 1, with the application device removed,

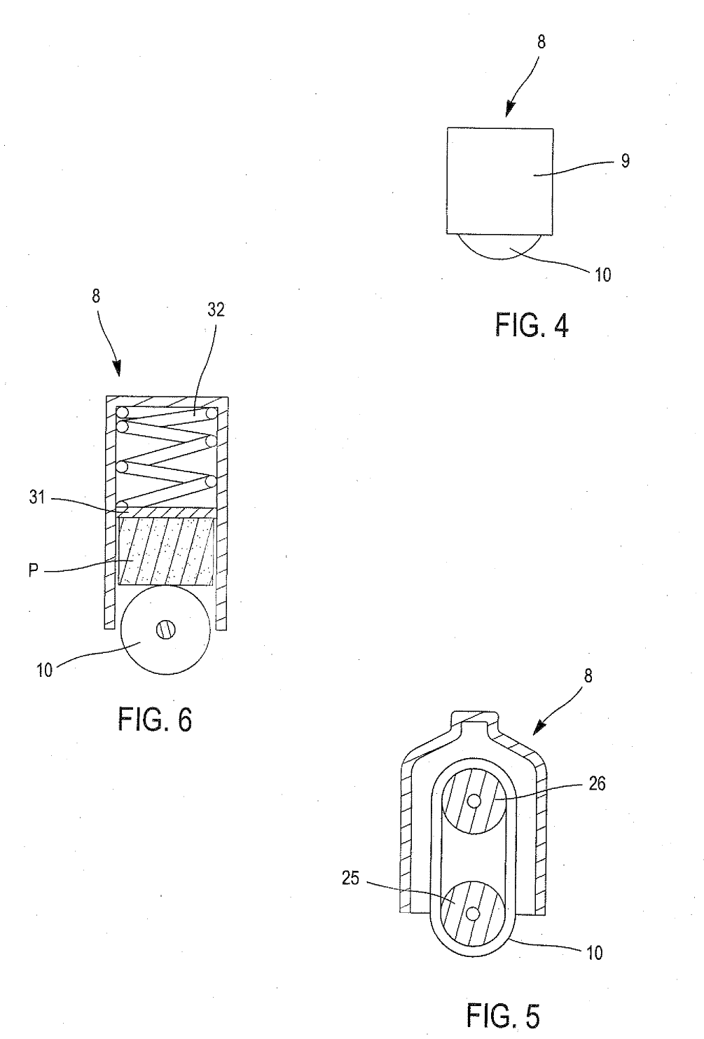

FIG. 4 is a schematic top view of an embodiment variant of the application member, and

FIGS. 5 and 6 represent, in partial and schematic longitudinal cross section, two other variants of application devices.

The conditioning and application device 1 shown in FIGS. 1, 2 and/or 3 comprises a case 2 comprising a base and a lid 4 that is mobile relative to the base, this lid 4 possibly comprising a transparent window 5 to be able to see the contents of the case when it is closed.

The base 3 comprises a housing 6 receiving a cake of product P and a housing 7 housing an application device 8, which serves to take up the product P and apply it to human keratin materials.

The application device 8 comprises a handle 9 and an application member 10 that can rotate relative to the handle 9, for example as illustrated about an axis of rotation X that may be oriented perpendicular to the longitudinal axis of the handle. This handle may have a generally flattened shape, parallel to a plane containing the axis of rotation X.

The application member 10 may be in the form of a roll, the application surface of which may be cylindrical in revolution about the axis X.

The application surface may be defined by any material that is capable of retaining the product and applying it, for example an elastomeric material or a foam, with open or closed cells, or a flocked membrane. The material defining the application surface may be compressible.

The length of the application member 10 may correspond substantially to the width w of the product cake. This product cake may have a rectangular shape, as illustrated.

The housing 7 receiving the application device 8 may have a bottom 14 provided with a grille 14 and may comprise a slope 16 that keeps the handle 9 slightly inclined upwards and towards the side of the case, so as to make it easy for the user to hold it. A recess 18 may emerge into the housing 7 so as to facilitate the engagement of a finger for gripping handle 9.

The housing 6 into which is poured the product P may be of parallelepiped shape or may have a rounded concave bottom that is a cylinder portion, with a generatrix parallel to the axis of rotation X when the application device 8 is moved over the surface of the product cake in order to take up product. This may avoid the presence of corners in which the product might accumulate and not be taken up.

The case may comprise a clasp 20, which may be of any type.

To use the application device 8, the user can move the application member 10 one or more times over the product cake P by rolling it over the surface of the product cake, and then apply it to the keratin material to be treated, for example the skin, by passing it one or more times over the area in which the product is to be deposited.

During the movement over the skin, the application member 10 can roll without sliding.

The invention is not limited to an application member that is in the form of a roll rotating about an axis of rotation.

For example, as illustrated in FIG. 4, the application member 10 may be in the form of an applicator ball 10, which may be held in a cage, not shown, integrally attached to a handle 9. In the example of FIG. 4, the application member 10 may rotate about all axes in its cage.

In one variant shown in FIG. 5, the application member 10 may also be defined by an application strip that can rotate about two rolls 25 and 26 with parallel axes of rotation. The portion of the strip 10 serving for the application can move on contact with the skin, when the application device 10 is moved in contact therewith to apply the product.

In one variant, not shown, the axes of rotation of the rolls 25 and 26 are arranged such that the portion of the strip 10 that comes into contact with the keratin materials to apply the product is not in contact with only one roll, but between the two rolls.

In the variant shown in FIG. 6, the product P is housed in a case that remains integrally attached to the application device 8 during the application of the product. The product P is, for example, in the form of a cake integrally attached to a dish 31, which is brought to bear against the application member 10 by a spring 32, for example of coil type as illustrated. In the example of FIG. 6, the application member 10 can take up product on one side while applying it on the other.

In variants not shown, the application device 8 may be equipped with a brake which makes it possible to brake, or even immobilize, the rotation of the application member 10 relative to the handle.

Composition

As stated above, the composition according to the invention is a solid composition.

The hardness measurement is performed using a TA-XT2 texture analyser sold by the company Rheo.

The hardness is likened to the compression force (in grams) measured during the penetration at 20° C. of a stainless-steel cylinder 3 mm in diameter to a depth of 2 mm and at a speed of 1 mm/second.

In the case of a cast product, the composition will preferably have a hardness of greater than or equal to 60 g, in particular between 60 g and 1000 g, preferably between 60 and 600 g and even more preferentially between 60 and 350 g.

According to one particular embodiment, a composition according to the invention advantageously has a hardness of less than or equal to 350 g, in particular less than or equal to 300 g, more particularly less than or equal to 250 g, especially ranging from 60 to 250 g.

Advantageously, a solid composition according to the invention is easily taken up using an application member as described previously.

The solid composition may be in an anhydrous form or in the form of a solid emulsion.

It should be noted that a solid emulsion does not flow under its own weight at room temperature, as opposed to a standard emulsion, and it is characterized especially by the presence of wax(es) in the liquid fatty phase.

The composition according to the invention may advantageously be an anhydrous composition, i.e. a composition containing less than 2% by weight of water or even less than 0.5% of water, especially free of water, the water not being added during the preparation of the composition but corresponding to the residual water provided by the mixed ingredients.

According to one particular embodiment, a composition according to the invention is in the form of an anhydrous cast product.

A cosmetic composition according to the invention thus comprises a liquid fatty phase and at least one wax especially in compositions in the form of a cast product, and may also comprise an aqueous phase, preferably in a proportion of at least 5% by weight, especially for products cast in the form of a solid emulsion.

A composition according to the invention may advantageously have a melting point ranging from 30 to 120° C., in particular from 40 to 100° C., in particular measured by DSC with a released energy ranging from −0.2 to 1 W/g.

This characterization by differential scanning calorimetry (DSC) may be performed with a DSC Q100 V9.4 BUILD 287 machine. The measurement is performed in temperature ramps from −20° C. to 120° C., over three melting cycles with a heating rate of 10° C./minute and of crystallization from 120 to −20° C. with a cooling rate of 10° C./minute for the first two cycles, and a cooling rate of 5° C./minute for the third cycle, with 6 mg of sample of the composition to be characterized.

On the third cycle, the variation of the difference in power absorbed by the empty crucible and by the crucible containing the sample is measured as a function of the temperature. The melting point is the temperature value corresponding to the top of the peak of the curve representing the variation of the difference in power absorbed as a function of the temperature.

Fatty Phase

The composition according to the invention comprises at least one liquid fatty phase and at least one wax.

In particular, a composition of the invention comprises at least one oil as mentioned below.

The term “oil” means any fatty substance that is in liquid form at room temperature (20-25° C.) and at atmospheric pressure.

Advantageously, a composition of the invention comprises at least one polar oil.

For the purposes of the present invention, the term “polar oil” means an oil whose solubility parameter at 25° C., δa, is other than 0 (J/cm³)^{1/2}.

The polar oil may be a volatile or non-volatile hydrocarbon-based, silicone and/or fluoro oil.

In particular, the polar oil will be present in the said composition in a content ranging from 5% to 50% by weight relative to the total weight of the composition, preferably ranging from 7% to 30% by weight, in particular ranging from 10% to 27% by weight relative to the total weight of the said composition.

A person skilled in the art will know how to select, from the list of volatile and non-volatile oils described below, the polar oils that may be used in the present invention.

In the case of a cast product, the content of liquid fatty phase may range from 10% to 80% by weight, preferably from 20% to 60% by weight, in particular from 25% to 50% by weight relative to the total weight of the said composition.

The oils may be present in the liquid fatty phase in a content ranging from 1% to 100% by weight, preferably ranging from 10% to 98% by weight and preferentially ranging from 25% to 95% by weight.

The oil(s) may be chosen especially from hydrocarbon-based, silicone and fluoro oils.

For the purposes of the present invention, the term:
“silicone oil” means an oil comprising at least one silicon atom and especially at least one Si—O group,
“fluoro oil” means an oil comprising at least one fluorine atom, and
“hydrocarbon-based oil” means an oil mainly containing hydrogen and carbon atoms.

The oils may optionally comprise oxygen, nitrogen, sulfur and/or phosphorus atoms, for example in the form of hydroxyl or acid radicals.

They may be of animal, plant, mineral or synthetic origin.

The oils may be volatile or non-volatile.

For the purposes of the present invention, the term “volatile oil” means an oil (or non-aqueous medium) that is capable of evaporating on contact with the skin in less than one hour, at room temperature and at atmospheric pressure. The volatile oil is a volatile cosmetic oil, which is liquid at room temperature, especially having a non-zero vapour pressure, at room temperature and at atmospheric pressure, in particular having a vapour pressure ranging from 0.13 Pa to 40 000 Pa (10⁻³ to 300 mmHg), preferably ranging from 1.3 Pa to 13 000 Pa (0.01 to 100 mmHg) and preferentially ranging from 1.3 Pa to 1300 Pa (0.01 to 10 mmHg).

For the purposes of the present invention, the term “non-volatile oil” means an oil having a vapour pressure of less than 0.13 Pa.

Volatile Oils

The volatile oils may be chosen from hydrocarbon-based oils containing from 8 to 16 carbon atoms, and especially C₈-C₁₆ branched alkanes (also known as isoparaffins), for instance isododecane (also known as 2,2,4,4,6-pentamethylheptane), isodecane and isohexadecane, for example the oils sold under the trade names Isopar® and Permethyl®.

Volatile oils that may also be used include volatile silicones, for instance volatile linear or cyclic silicone oils, especially those with a viscosity of less than or equal to 8 centistokes (cSt) (8×10⁻⁶ m²/s) and especially containing 2 to 10 silicon atoms and in particular 2 to 7 silicon atoms, these silicones optionally comprising alkyl or alkoxy groups containing 1 to 10 carbon atoms. Mention may be made of dimethicones with a viscosity of and 6 cSt, octamethylcyclotetrasiloxane, decamethylcyclopentasiloxane, dodecamethylcyclohexasiloxane, heptamethylhexyltrisiloxane, heptamethyloctyltrisiloxane, hexamethyldisiloxane, octamethyltrisiloxane, decamethyltetrasiloxane and dodecamethylpentasiloxane, and mixtures thereof.

Volatile fluoro oils such as nonafluoromethoxybutane and perfluoromethylcyclopentane, and mixtures thereof, may also be used.

Advantageously, the volatile oil may be present in a content ranging from 0.1% to 50% by weight, preferably ranging from 1% to 30% by weight and preferentially ranging from 5% to 25% by weight relative to the total weight of the composition.

Non-Volatile Oils

The non-volatile oils may be chosen especially from non-volatile hydrocarbon-based, fluoro and/or silicone oils.

Non-volatile hydrocarbon-based oils that may especially be mentioned include:
hydrocarbon-based oils of animal origin, such as perhydrosqualene
hydrocarbon-based oils of plant origin, such as phytostearyl esters, such as phytostearyl oleate, phytostearyl isostearate and lauroyl/octyldodecyl/phytostearyl glutamate (Ajinomoto, Eldew PS203); triglycerides formed from fatty acid esters of glycerol, in particular whose fatty acids may have chain lengths ranging from C₄ to C₃₆ and especially from C₁₈ to C₃₆, these oils possibly being linear or branched, and saturated or unsaturated; these oils may especially be heptanoic or octanoic triglycerides, shea oil, alfalfa oil, poppy oil, pumpkin oil, millet oil, barley oil, quinoa oil, rye oil, candlenut oil, passionflower oil, shea butter oil, aloe oil, sweet almond oil, peach stone oil, groundnut oil, argan oil, avocado oil, baobab oil, borage oil, broccoli oil, calendula oil, camellina oil, canola oil, carrot oil, safflower oil, hemp oil, rapeseed oil, cottonseed oil, coconut oil, marrow seed oil, wheatgerm oil, jojoba oil, lily oil, macadamia oil, corn oil, meadowfoam oil, St-John's wort oil, monoi oil, hazelnut oil, apricot kernel oil, walnut oil, olive oil, evening primrose oil, palm oil, blackcurrant pip oil, kiwi seed oil, grape seed oil, pistachio oil, pumpkin oil, quinoa oil, musk rose oil, sesame oil, soybean oil, sunflower oil, castor oil and watermelon oil, and mixtures thereof, or alternatively caprylic/capric acid triglycerides, such as those sold by the company Stearineries Dubois or those sold under the names Miglyol 810®, 812® and 818® by the company Dynamit Nobel;
linear or branched hydrocarbons of mineral or synthetic origin, such as liquid paraffins and derivatives thereof, petroleum jelly, polydecenes, polybutenes, hydrogenated polyisobutene such as Parleam, and squalane;
synthetic ethers containing from 10 to 40 carbon atoms;
synthetic esters, for instance the oils of formula R₁COOR₂, in which R₁ represents a linear or branched fatty acid residue containing from 1 to 40 carbon atoms and R₂ represents a hydrocarbon-based chain, which is especially branched, containing from 1 to 40 carbon atoms, on condition that R₁+R₂≧10. The esters may be chosen especially from esters of alcohol and of fatty acid, for instance cetostearyl octanoate, isopropyl alcohol esters, such as isopropyl myristate, isopropyl palmitate, ethyl palmitate, 2-ethylhexyl palmitate, isopropyl stearate, isopropyl isostearate, isostearyl isostearate, octyl stearate, hydroxylated esters, for instance isostearyl lactate, octyl hydroxystearate, diisopropyl adipate, heptanoates, and especially isostearyl heptanoate, octanoates, decanoates or ricinoleates of alcohols or of polyalcohols, for instance propylene glycol dioctanoate, cetyl octanoate, tridecyl octanoate, 2-ethylhexyl 4-diheptanoate, 2-ethylhexyl palmitate, alkyl benzoate, polyethylene glycol diheptanoate, propylene glycol 2-diethylhexanoate and mixtures thereof, C₁₂-C₁₅ alkyl benzoates, hexyl laurate, neopentanoic acid esters, for instance isodecyl neopentanoate, isotridecyl neopentanoate, isostearyl neopentanoate or octyldodecyl neopentanoate, isononanoic acid esters, for instance isononyl isononanoate, isotridecyl isononanoate and octyl isononanoate, and hydroxylated esters, for instance isostearyl lactate and diisostearyl malate;
polyol esters and pentaerythritol esters, for instance dipentaerythrityl tetrahydroxystearate/tetraisostearate;
esters of diol dimers and of diacid dimers, such as Lusplan DD-DA5® and Lusplan DD-DA7®, sold by the company Nippon Fine Chemicals and described in patent application US 2004/175 338;
copolymers of diol dimer and of diacid dimer and esters thereof, such as dilinoleyl diol dimer/dilinoleic dimer copolymers, and esters thereof, for instance Plandool-G;
copolymers of polyols and of diacid dimers, and esters thereof, such as Hailuscent ISDA, or the dilinoleic acid/butanediol copolymer;
fatty alcohols that are liquid at room temperature, with a branched and/or unsaturated carbon-based chain containing from 12 to 26 carbon atoms, for instance 2-octyldodecanol, isostearyl alcohol, oleyl alcohol, 2-hexyldecanol, 2-butyloctanol and 2-undecylpentadecanol;
C₁₂-C₂₂ higher fatty acids, such as oleic acid, linoleic acid and linolenic acid, and mixtures thereof; and
dialkyl carbonates, the two alkyl chains possibly being identical or different, such as dicaprylyl carbonate sold under the name Cetiol CC® by Cognis;
oils of high molar mass, in particular having a molar mass ranging from about 400 to about 10 000 g/mol, in particular from about 650 to about 10 000 g/mol, in particular from about 750 to about 7500 g/mol and more particularly ranging from about 1000 to about 5000 g/mol. As oil of high molar mass that may be used in the present invention, mention may be made especially of oils chosen from:
   lipophilic polymers,
   linear fatty acid esters with a total carbon number ranging from 35 to 70,
   hydroxylated esters,
   aromatic esters,
   C₂₄-C₂₈ esters of branched fatty alcohols or fatty acids,
   silicone oils,
   oils of plant origin,
   and mixtures thereof,
fluoro oils that are optionally partially hydrocarbon-based and/or silicone-based, for instance fluorosilicone oils, fluoro polyethers, and fluorosilicones as described in document EP-A-847 752;
silicone oils such as linear or cyclic, non-volatile polydimethylsiloxanes (PDMS); polydimethylsiloxanes comprising alkyl, alkoxy or phenyl groups, pendent or at the end of a silicone chain, these groups containing from 2 to 24 carbon atoms; phenyl silicones, for instance phenyl trimethicones, phenyl dimethicones, phenyltrimethylsiloxydiphenylsiloxanes, diphenyl dimethicones, diphenyl methyl diphenyl trisiloxanes and 2-phenyl ethyl trimethyl siloxysilicates, and
mixtures thereof.

Advantageously, the non-volatile oil may be present in a content ranging from 0.1% to 50% by weight, preferably ranging from 1% to 30% by weight and preferentially ranging from 5% to 25% by weight relative to the total weight of the composition.

Advantageously, a composition according to the invention comprises at least one polar oil, in particular of ester oil type. More particularly, the ester oil is a monoester.

The ester oil may preferably be chosen from the monoesters of formula R₁COOR₂ in which R₁ represents a linear or branched hydrocarbon-based chain comprising from 4 to 40 carbon atoms, preferably from 4 to 30 carbon atoms and preferentially from 7 to 20 carbon atoms, and R₂ represents a branched hydrocarbon-based chain containing from 3 to 40 carbon atoms, preferably from 10 to 30 carbon atoms and preferentially from 16 to 26 carbon atoms.

Ester oils that are most particularly suitable for use include isodecyl neopentanoate; isocetyl octanoate; isononyl isononanoate; isodecyl isononanoate; tridecyl isononanoate; hexyl laurate, 2-hexyldecyl laurate; isopropyl myristate, isocetyl myristate, isotridecyl myristate, 2-octyldodecyl myristate; isopropyl palmitate, 2-ethylhexyl palmitate, isooctyl palmitate, isocetyl palmitate, isodecyl palmitate, isostearyl palmitate, 2-octyldecyl palmitate; isopropyl isostearate, 2-octyldodecyl stearate, isostearyl isostearate; 2-octyldodecyl erucate; and mixtures thereof.

More preferably, an ester oil chosen from isodecyl neopentanoate; isocetyl octanoate; isononyl isononanoate; isostearyl isostearate; and mixtures thereof, will be used.

The polar oil, in particular of ester oil type, may be present in the composition according to the invention in a content ranging from 5% to 50% by weight, preferably ranging from 7% to 30% by weight and preferentially ranging from 10% to 27% by weight relative to the total weight of the composition.

Advantageously, a composition according to the invention comprises at least one ester oil, the ester oil(s) being present in a total content of greater than or equal to 20% by weight relative to the total weight of said composition, in particular from 20% to 45% by weight, preferably from 20% to 40% and more preferentially from 25% to 35% by weight relative to the total weight of said composition.

According to one particularly preferred embodiment, a composition according to the invention comprises at least isononyl isononanoate and/or 2-ethylhexyl palmitate, preferably at least isononyl isononanoate.

Advantageously, the isononyl isononanoate is present in a content ranging from 5% to 50% by weight, preferably from 10% to 40% by weight, more preferentially from 20% to 35% by weight and especially from 25% to 30% by weight relative to the total weight of the composition.

According to yet another particular embodiment, a composition according to the invention comprises one or more ester oils, and one or more pigments, in an ester oil(s)/pigment(s) weight ratio of greater than or equal to 1, in particular ranging from 1 to 2 and preferably from 1 to 1.5.

In particular, the presence of at least one ester oil as described previously, and more particularly of at least isononyl isononanoate, makes it possible to improve the dispersion of the pigments in the solid composition according to the invention. The use of such a composition, coupled with the specific application device of the cosmetic makeup and/or care assembly according to the invention, advantageously makes it possible to obtain a uniform makeup effect, in particular in terms of the colour effect.

Silicone Resins

Besides at least one oil, the fatty phase may also advantageously comprise at least one silicone resin.

Silicone resins are products of hydrolysis and polycondensation of siloxane mixtures of formula (R)₃SiOCH₃ and Si(OCH₃)₄, R representing an alkyl group containing from 1 to 6 carbon atoms.

These silicone resins are known or may be prepared according to known methods. Among the commercial silicone resins that may be used, mention may be made, for example, of those sold under the names DC 593 (Dow Corning) or SS 4230 (General Electric).

The silicone resin may be present in a content ranging from 0.1% to 40% and in particular from 2% to 30% by weight relative to the total weight of the composition.

Waxes

A composition according to the invention will comprise at least one wax, especially as agent for structuring the liquid fatty phase.

For the purposes of the present invention, the term “wax” means a lipophilic compound, which is solid at room temperature (25° C.), with a reversible solid/liquid change of state, having a melting point of greater than or equal to 30° C., which may be up to 200° C. and especially up to 120° C.

In particular, the waxes that are suitable for use in the invention may have a melting point of greater than or equal to 45° C. and in particular greater than or equal to 55° C.

By bringing a wax to the liquid state (melting), it is possible to make it miscible with the oils that may be present and to form a microscopically homogeneous mixture, but on reducing the temperature of the mixture to room temperature, recrystallization of the wax in the oils of the mixture is obtained.

For the purposes of the invention, the melting point corresponds to the temperature of the most endothermic peak observed by thermal analysis (DSC) as described in standard ISO 11357-3, 1999. The melting point of the wax may be measured using a differential scanning calorimeter (DSC), for example the calorimeter sold under the name MDSC 2920 by the company TA Instruments.

The measuring protocol is as follows:

A sample of 5 mg of wax placed in a crucible is subjected to a first temperature rise ranging from −20° C. to 100° C., at a heating rate of 10° C./minute, it is then cooled from 100° C. to −20° C. at a cooling rate of 10° C./minute and is finally subjected to a second temperature increase ranging from −20° C. to 100° C. at a heating rate of 5° C./minute. During the second temperature increase, the variation of the difference in power absorbed by the empty crucible and by the crucible containing the sample of wax is measured as a function of the temperature. The melting point of the compound is the temperature value corresponding to the top of the peak of the curve representing the variation in the difference in absorbed power as a function of the temperature.

The wax may especially have a hardness ranging from 0.05 MPa to 15 MPa and preferably ranging from 6 MPa to 15 MPa or even from 6.5 MPa to 15 MPa. The hardness is determined by measuring the compression force measured at 20° C. using a texturometer sold under the name TA-TX21 by the company Rheo, equipped with a stainless-steel cylinder 2 mm in diameter travelling at a measuring speed of 0.1 mm/second and penetrating into the wax to a penetration depth of 0.3 mm.

A composition according to the invention advantageously contains at least two waxes of different polarities.

Advantageously, one of these waxes is an apolar wax, especially as defined hereinbelow, and may have a melting point ranging from 50 to 100° C., and the other wax is a polar wax, especially as defined hereinbelow, and may have a melting point ranging from 40 to 100° C.

According to one embodiment variant, these two types of wax have the same melting point.

Polar Wax

For the purposes of the present invention, the term “polar” wax means a wax whose solubility parameter at 25° C., δa, is other than 0 (J/cm³)^{1/2.}

In particular, the term “polar” wax means a wax whose chemical structure is formed essentially from, or even constituted of, carbon and hydrogen atoms, and comprising at least one strongly electronegative heteroatom such as an oxygen, nitrogen or phosphorus atom.

The definition and calculation of the solubility parameters in the three-dimensional Hansen solubility space are described in the article by C. M. Hansen: “The three dimensional solubility parameters” J. Paint Technol. 39, 105 (1967).

According to this Hansen space:

δ_{D} characterizes the London dispersion forces derived from the formation of induced dipoles during molecular impacts;
δₚ characterizes the Debye interaction forces between permanent dipoles and the Keesom interaction forces between induced dipoles and permanent dipoles;
δₕ characterizes the specific interaction forces (such as hydrogen bonding, acid/base, donor/acceptor, etc.);
δₐ is determined by the equation: δₐ=(δₚ²+δₕ²)^{1/2}.

The parameters δₚ, δₕ, δ_{D} and δₐ are expressed in (J/cm³)^{1/2}.

The polar waxes that may be used in the compositions according to the invention are chosen from waxes that are solid at room temperature, of animal, plant, mineral and/or synthetic origin, and mixtures thereof.

The waxes may especially be hydrocarbon-based or silicone waxes.

The term “silicone wax” means an oil comprising at least one silicon atom and especially comprising Si—O groups.

The term “hydrocarbon-based wax” means a wax formed essentially from, or even constituted of, carbon and hydrogen atoms, and optionally oxygen and nitrogen atoms, and not containing any silicon or fluorine atoms. It may contain alcohol, ester, ether, carboxylic acid, amine and/or amide groups.

Silicone Waxes

As illustrations of these waxes, mention may be made of substituted linear polysiloxanes, for example polyether silicone waxes, alkyl or alkoxy dimethicones containing from 16 to 45 carbon atoms, and alkyl methicones such as the C₃₀-C₄₅ alkyl methicone sold under the trade name AMS C 30 by Dow Corning.

The composition according to the invention may especially comprise substituted polysiloxanes, preferably with a low melting point, for example substituted linear polysiloxanes constituted essentially (apart from the end groups) of units of formulae (II) and (III), in the respective mole proportions m and n:

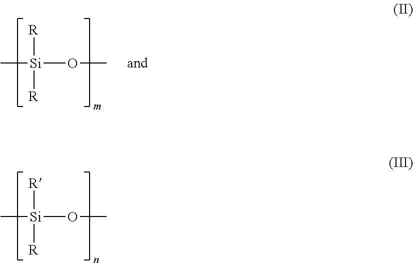

in which:
   each substituent R is defined as above,
   each R′ independently represents a linear or branched, saturated or unsaturated alkyl containing from 6 to 30 carbon atoms, or a group —X—R″, each X independently representing:
      —O—,
      —(CH₂)ₐ—O—CO—,
      —(CH₂)_{b}—CO—O—,
a and b independently represent numbers possibly ranging from 0 to 6, and
each R″ independently represents an optionally unsaturated alkyl group containing from 6 to 30 carbon atoms,
   m is a number possibly ranging from 0 to 400 and in particular from 0 to 100,
   n is a number possibly ranging from 1 to 200 and in particular from 1 to 100, the sum (m+n) being less than 400 and in particular less than or equal to 100.

These silicone waxes are known or may be prepared according to the known methods. Among the commercial silicone waxes of this type, mention may be made especially of those sold under the names Abilwax 9800, 9801 or 9810 (Goldschmidt), KF910 and KF7002 (Shin-Etsu) or 176-1118-3 and 176-11481 (General Electric).

The silicone waxes that may be used may also be chosen from the compounds of formula (IV):

R₁—Si(CH₃)₂—O—[Si(R)₂—O—]—Si(CH₃)₂—R₂ (IV)
in which:
R is defined as above,
R₁ represents an alkyl group containing from 1 to 30 carbon atoms, an alkoxy group containing from 6 to 30 carbon atoms or a group of formula:

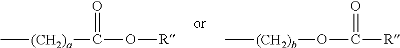

R₂ represents an alkyl group containing from 6 to 30 carbon atoms, an alkoxy group containing from 6 to 30 carbon atoms or a group of formula:

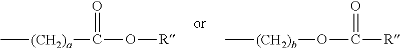

a and b representing a number from 0 to 6,
R″ being an alkyl containing from 6 to 30 carbon atoms,
and z is a number possibly ranging from 1 to 100.

Among the silicone waxes of formula (IV), which are known products or which may be prepared according to known methods, mention will be made especially of the following commercial products: Abilwax 2428, 2434 and 2440 (Goldschmidt) or VP 1622 and VP 1621 (Wacker).

Such a wax may be present in the composition especially in the form of a cast product according to the invention in a content ranging from 1% to 15% by weight, preferably ranging from 2% to 10% by weight and preferentially ranging from 2% to 5% by weight relative to the total weight of the composition.

Hydrocarbon-Based Waxes

Preferably, the polar wax is hydrocarbon-based.

As illustrations of hydrocarbon-based waxes that are suitable for use in the invention, mention may be made of beeswax, lanolin wax, rice bran wax, carnauba wax, candelilla wax, shellac wax, montan wax, orange wax, lemon wax, laurel wax, hydrogenated jojoba oil and olive wax.

According to one particular mode, carnauba wax will be used.

According to one preferred embodiment, the polar wax is an ester wax.

According to the invention, the term “ester wax” means a wax comprising at least one ester function.

It is especially possible to use as ester wax:
ester waxes such as:
i) the waxes of formula R₁COOR₂ in which R₁ and R₂ represent linear, branched or cyclic aliphatic chains in which the number of atoms ranges from 10 to 50, which may contain a heteroatom such as O, N or P, and whose melting point ranges from 25 to 120° C. In particular, a C₂₀-C₄₀ alkyl (hydroxystearyloxy)stearate (the alkyl group containing from 20 to 40 carbon atoms), alone or as a mixture, or a C₂₀-C₄₀ alkyl stearate, may be used as ester wax. Such waxes are especially sold under the names Kester Wax K 82 P®, Hydroxypolyester K 82 P®, Kester Wax K 80 P® and Kester Wax K82H by the company Koster Keunen.

It is also possible to use an ester of polyethylene glycol and of montanic acid (octacosanoic acid), such as the wax Licowax KPS Flakes (INCI name: glycol montanate) sold by the company Clariant.
ii) di(1,1,1-trimethylolpropane) tetrastearate, sold under the name Hest 2T-4S® by the company Heterene,
iii) waxes of diesters of a dicarboxylic acid, of general formula R³—(—OCO—R⁴—COO—R⁵), in which R³ and R⁵ are identical or different, preferably identical, and represent a C₄-C₃₀ alkyl group (alkyl group comprising from 4 to 30 carbon atoms) and R⁴ represents a linear or branched C₄-C₃₀ aliphatic group (alkyl group comprising from 4 to 30 carbon atoms), which may or may not contain one or more unsaturations, and is preferably linear and unsaturated,
iv) waxes obtained by hydrogenation of castor oil esterified with cetyl alcohol, sold under the names Phytowax ricin 16L64® and 22L73® by the company Sophim. Such waxes are described in patent application FR-A-2 792 190.

According to another embodiment, the polar wax may be an alcohol wax. According to the invention, the term “alcohol wax” means a wax comprising at least one alcohol function, i.e. comprising at least one free hydroxyl (OH) group.

An example of an alcohol wax that may be mentioned is the mixture of linear long-chain (C₃₀-C₅₀) fatty alcohol and of hydrocarbon containing the same carbon number (80/20), such as the wax Performacol 550-L Alcohol from New Phase Technologies.

According to one particular mode, the mixture of linear long-chain (C₃₀-C₅₀) fatty alcohol and of hydrocarbon containing the same carbon number (80/20), such as the wax Performacol 550-L Alcohol from New Phase Technologies, will be used as ester wax.

Preferably, the composition may comprise a content of polar wax, in particular of hydrocarbon-based polar wax and more particularly of hydrocarbon-based ester wax, ranging from 0.1% to 50% by weight of wax relative to the total weight of the composition, better still from 1% to 30% by weight and in particular from 1% to 20% by weight relative to the total weight of the composition.

Apolar Wax

For the purposes of the present invention, the term “apolar wax” means a wax whose solubility parameter at 25° C. as defined below, Sₐ, is equal to 0 (J/cm³)^{1/2}. Apolar waxes are, in particular, hydrocarbon-based waxes constituted solely of carbon and hydrogen atoms and free of heteroatoms such as N, O and P.

In particular, the term “apolar wax” means a wax constituted solely of apolar wax, rather than a mixture also comprising other types of wax that are not apolar waxes.

As illustrations of apolar waxes that are suitable for use in the invention, mention may be made especially of hydrocarbon-based waxes, for instance microcrystalline waxes, paraffin waxes, ozokerites and polyethylene waxes.

Polyethylene waxes that may be mentioned include Performalene 500-L Polyethylene and Performalene 400 Polyethylene sold by New Phase Technologies.

An ozokerite that may be mentioned is Ozokerite Wax SP 1020 P.

As microcrystalline waxes that may be used, mention may be made of Multiwax W 445® sold by the company Sonneborn, and Microwax HW® and Base Wax 30540® sold by the company Paramelt.

As microwaxes that may be used in the compositions according to the invention as apolar wax, mention may be made especially of polyethylene microwaxes such as those sold under the names Micropoly 200®, 220®, 220L® and 250S® by the company Micro Powders.

According to one particular mode, a polyethylene wax will be used.

According to another mode, a microcrystalline wax will be used.

According to one particular embodiment, the composition according to the invention contains a hydrocarbon-based apolar wax and a hydrocarbon-based polar wax.

In particular, the composition comprises at least one hydrocarbon-based polar wax chosen from beeswax, rice bran wax, carnauba wax, hydrogenated jojoba oil, an alcohol wax and a hydrocarbon-based apolar wax chosen from a polyethylene wax and a microcrystalline wax, and mixtures thereof.

According to one preferred embodiment, the composition according to the invention contains at least one alcohol wax and one polyethylene wax.

According to another preferred embodiment, the composition according to the invention contains at least one carnauba wax and at least one microcrystalline wax.

According to yet another preferred embodiment, the composition according to the invention contains at least one hydrogenated jojoba oil (also named hydrogenated jojoba wax) and a polyethylene wax.

Preferably, the composition may contain from 0.1% to 50% by weight of waxes relative to the total weight of the composition, better still from 1% to 30% by weight or alternatively between 3% and 20% by weight relative to the total weight of the composition.

Advantageously, a composition according to the invention comprises from 4% to 15% by weight of waxes relative to the total weight of the said composition, preferably from 5% to 10% by weight and especially from 7% to 8% by weight of waxes relative to the total weight of the said composition.

According to one particularly preferred embodiment, a composition according to the invention comprises at least one hard wax as defined below.

For the purposes of the invention, the term “hard” wax means a wax with a hardness of greater than or equal to 6 MPa, especially greater than or equal to 6.5 MPa, especially ranging from 6 MPa to 30 MPa, or even from 6.5 MPa to 30 MPa, and in particular ranging from 6 MPa to 15 MPa, or even from 6.5 MPa to 15 MPa.

Examples of hard waxes according to the present invention that may especially be mentioned include carnauba wax, microcrystalline waxes, polyethylene waxes, candelilla wax, hydrogenated jojoba oil (also named hydrogenated jojoba wax), rice bran wax, ozokerites, Chinese insect wax, shellac wax, sumac wax, ouricury wax and ceresin.

It is also possible to use the wax derived from hydrogenation of esterified olive oil with stearylalcohol sold under the name Phytowax Olive 18L57, or else waxes derived from hydrogenation of esterified castor oil with cetyl alcohol sold under the name Phytowax Ricin 16L64 and 22L73 by the company Sophim. Such waxes are described in patent application FR-A-2792190.

Preferably, a composition according to the invention comprises one or more hard wax(es) in a content of greater than or equal to 3% by weight relative to the total weight of the said composition, in particular greater than or equal to 4% by weight, especially ranging from 5% to 10% by weight and preferably from 7% to 8% by weight relative to the total weight of the said composition.

According to one particular embodiment, a composition according to the invention comprises at least two “hard” waxes, preferably of different polarities.

In particular, a composition according to the invention comprises at least one apolar hard wax and one polar hard wax.

Pasty Compound

A composition according to the invention may also comprise at least one pasty compound.

The presence of a pasty compound may make it possible to advantageously impart improved comfort during the deposition of a composition of the invention.

Such a compound may be advantageously chosen from lanolin and derivatives thereof; polymer or non-polymer silicone compounds; polymer or non-polymer fluor compounds; vinyl polymers, especially olefin homopolymers; olefin copolymers; hydrogenated diene homopolymers and copolymers; linear or branched oligomers, which are homopolymers or copolymers of alkyl (meth)acrylates preferably containing a C₈-C₃₀ alkyl group; oligomers, which are homopolymers and copolymers of vinyl esters containing C₈-C₃₀ alkyl groups; oligomers, which are homopolymers and copolymers of vinyl ethers containing C₈-C₃₀ alkyl groups; liposoluble polyethers resulting from the polyetherification between one or more C₂-C₁₀₀ and preferably C₂-C₅₀ diols; fatty acid or fatty alcohol esters; and mixtures thereof.

Among the esters, mention may be made especially of:
esters of a glycerol oligomer, especially diglycerol esters, for instance polyglyceryl-2 triisostearate, condensates of adipic acid and of glycerol, for which some of the hydroxyl groups of the glycerols have reacted with a mixture of fatty acids such as stearic acid, capric acid, stearic acid and isostearic acid, and 12-hydroxystearic acid, especially such as the product sold under the brand name Softisan 649 by the company Sasol, or such as bis(diglyceryl) poly(2-acyladipate); arachidyl propionate sold under the brand name Waxenol 801 by Alzo; phytosterol esters; fatty acid triglycerides and derivatives thereof, such as hydrogenated cocoglycerides; non-crosslinked polyesters resulting from polycondensation between a linear or branched C₄-C₅₀ dicarboxylic acid or polycarboxylic acid and a C₂-C₅₀ diol or polyol; aliphatic esters of an ester resulting from the esterification of an aliphatic hydroxycarboxylic acid with an aliphatic carboxylic acid; polyesters resulting from the esterification, with a polycarboxylic acid, of an ester of an aliphatic hydroxycarboxylic acid, the said ester comprising at least two hydroxyl groups, such as the products Risocast DA-H® and Risocast DA-L®; and mixtures thereof.

The pasty compound(s) may be present in a composition of the invention in a content ranging from 0.1% to 30% by weight and more preferably from 0.5% to 20% by weight of agents relative to the total weight of the composition.

Thickeners

A composition of the invention may also comprise one or more thickeners or gelling agents.

Hydrophilic Thickener or Gelling Agent

A composition in the form of a cast product of solid emulsion type according to the invention may comprise a hydrophilic, i.e. water-soluble or water-dispersible, thickener or gelling agent.

Hydrophilic gelling agents that may be mentioned in particular include water-soluble or water-dispersible thickening polymers. These polymers may be chosen especially from: modified or unmodified carboxyvinyl polymers, such as the products sold under the name Carbopol (CTFA name: carbomer) by the company Goodrich; polyacrylates and polymethacrylates such as the products sold under the names Lubrajel and Norgel by the company Guardian or under the name Hispagel by the company Hispano Chimica; polyacrylamides; optionally crosslinked and/or neutralized 2-acrylamido-2-methylpropanesulfonic acid polymers and copolymers, for instance the poly(2-acrylamido-2-methylpropanesulfonic acid) sold by the company Clariant under the name Hostacerin AMPS (CTFA name: ammonium polyacryldimethyltauramide); crosslinked anionic copolymers of acrylamide and of AMPS, which are in the form of a W/O emulsion, such as those sold under the name Sepigel 305 (CTFA name: Polyacrylamide/C₁₃₋₁₄ Isoparaffin/Laureth-7) and under the name Simulgel 600 (CTFA name: Acrylamide/Sodium acryloyldimethyltaurate copolymer/Isohexadecane/Polysorbate 80) by the company SEPPIC; polysaccharide biopolymers, for instance xanthan gum, guar gum, carob gum, acacia gum, scleroglucans, chitin and chitosan derivatives, carrageenans, gellans, alginates, celluloses such as microcrystalline cellulose, carboxymethylcellulose, hydroxymethylcellulose and hydroxypropylcellulose; and mixtures thereof.

Lipophilic Thickener or Gelling Agent

It is also possible to use in the compositions of the invention, in particular in cast products, a lipophilic thickener or gelling agent, of mineral or organic type.

Examples of lipophilic thickeners or gelling agents that may be mentioned include modified clays such as modified magnesium silicate (Bentone gel VS38 from Rheox), modified hectorites such as hectorite modified with a C₁₀ to C₂₂ fatty acid ammonium chloride, for instance hectorite modified with distearyldimethylammonium chloride, for instance the product sold under the name Bentone 38V® by the company Elementis, the product sold under the name Bentone 38 CE by the company Rheox, or the product sold under the name Bentone Gel V5 5V by the company Elementis.

The polymeric organic lipophilic gelling agents are, for example, partially or totally crosslinked elastomeric organopolysiloxanes, of three-dimensional structure, for instance those sold under the names KSG6®, KSG16® and KSG18® by the company Shin-Etsu, Trefil E-505C® and Trefil E-506C® by the company Dow Corning, Gransil SR-CYC®, SR DMF10®, SR-DC556®, SR 5CYC Gel®, SR DMF 10 Gel and SR DC 556 Gel® by the company Grant Industries, and SF 1204® and JK 113® by the company General Electric; ethylcellulose, for instance the product sold under the name Ethocel® by the company Dow Chemical; polycondensates of polyamide type resulting from condensation between a dicarboxylic acid comprising at least 32 carbon atoms and an alkylenediamine and in particular ethylenediamine, in which the polymer comprises at least one terminal carboxylic acid group esterified or amidated with at least one saturated, linear monoalcohol or monoamine comprising from 12 to 30 carbon atoms, and in particular copolymers of ethylenediamine/stearyl dilinoleate such as the product sold under the name Uniclear 100 VG® by the company Arizona Chemical; galactomannans comprising from one to six and in particular from two to four hydroxyl groups per saccharide, substituted with a saturated or unsaturated alkyl chain, for instance guar gum alkylated with C₁-C₆ and in particular C₁-C₃ alkyl chains and mixtures thereof. Block copolymers of diblock, triblock or radial type of the polystyrene/polyisoprene or polystyrene/polybutadiene type such as those sold under the name Luvitol HSB® by the company BASF, of the polystyrene/copoly(ethylene-propylene) type such as those sold under the name Kraton® by the company Shell Chemical Co. or alternatively of the polystyrene/copoly(ethylene-butylene) type, mixtures of triblock and radial (star) copolymers in isododecane, such as those sold by the company Penreco under the name Versagel®, for instance the mixture of butylene/ethylene/styrene triblock copolymer and of ethylene/propylene/styrene star copolymer in isododecane (Versagel M 5960).

Among the lipophilic gelling agents that may be used in a cosmetic composition of the invention, mention may also be made of fatty acid esters of dextrin, such as dextrin palmitates, especially such as those sold under the names Rheopearl TL® and Rheopearl KL® by the company Chiba Flour, hydrogenated plant oils, such as hydrogenated castor oil, and fatty alcohols, in particular of C₈-C₂₆ and more particularly C₁₂-C₂₂, for instance myristyl alcohol, cetyl alcohol, stearyl alcohol or behenyl alcohol.

According to one embodiment, a composition of the invention especially in the form of a cast product may comprise thickeners in an active material content ranging from 0.01% to 40% by weight, especially from 0.1% to 20% by weight and more particularly from 0.3% to 15% by weight relative to the total weight of the composition.

Film-Forming Polymers

The compositions according to the invention may also comprise at least one film-forming polymer, especially for the compositions of the invention in the form of a cast product.

The term “polymer” means herein a compound containing at least two repeating units and preferably at least three repeating units.

The term “film-forming” polymer means a polymer that is capable, by itself or in the presence of an auxiliary film-forming agent, of forming a macroscopically continuous film on a support, especially on keratin materials.

Such a polymer or polymer mixture may be present in the composition in a content ranging from 0.1% to 30% by weight, preferably ranging from 0.3% to 25% by weight, preferably ranging from 0.5% to 20% by weight, preferentially ranging from 1% to 20% by weight and more preferentially ranging from 2% to 15% by weight, relative to the total weight of the composition.

In one embodiment, the film-forming organic polymer is at least one polymer chosen from the group comprising:
film-forming polymers that are soluble in an organic liquid medium, in particular liposoluble polymers, when the organic liquid medium comprises at least one oil;
film-forming polymers that are dispersible in an organic solvent medium, in particular polymers in the form of non-aqueous dispersions of polymer particles, preferably dispersions in silicone oils or hydrocarbon-based oils; in one embodiment, the non-aqueous polymer dispersions comprise polymer particles surface-stabilized with at least one stabilizer;
film-forming polymers in the form of aqueous dispersions of polymer particles, often referred to as “latices”; in this case, the composition comprises an aqueous phase; and
water-soluble film-forming polymers; in this case, the composition comprises an aqueous phase.

Among the film-forming polymers that may be used in the composition of the present invention, mention may be made of synthetic polymers, of radical type or of polycondensate type, polymers of natural origin, and mixtures thereof. Film-forming polymers that may be mentioned in particular include acrylic polymers, polyurethanes, polyesters, polyamides, polyureas, silicone polymers, silicone-grafted acrylic polymers, polyamide polymers and copolymers, and polyisoprenes.

They may more particularly be polyurethanes and/or polyamides.

It is understood that the amount of these additional compounds may be adjusted by a person skilled in the art so as not to harm the desired effect in the context of the present invention.

Other Polymers

The composition according to the invention may also contain an elastomer, especially a polyglycerolated silicone elastomer. By way of example, use is made of an elastomeric crosslinked organopolysiloxane that may be obtained by crosslinking addition reaction of a diorganopolysiloxane containing at least one hydrogen bonded to silicon and of polyglycerolated compounds containing ethylenically unsaturated groups, especially in the presence of a platinum catalyst.

Polyglycerolated silicone elastomers that may be used include those sold under the names KSG-710, KSG-810, KSG-820, KSG-830 and KSG-840 by the company Shin-Etsu.

The compositions according to the invention may also comprise an additional emulsifying silicone elastomer.

By way of example, use is made of polyoxyalkylenated elastomers as described especially in U.S. Pat. No. 5,236,986, U.S. Pat. No. 5,412,004, U.S. Pat. No. 5,837,793 and U.S. Pat. No. 5,811,487, the content of which is incorporated by reference.

Polyoxyalkylenated silicone elastomers that may be used include those sold under the names KSG-21, KSG-20, KSG-30, KSG-31, KSG-32, KSG-33, KSG-210, KSG-310, KSG-320, KSG-330, KSG-340 and X-226146 by the company Shin-Etsu, and DC9010 and DC9011 by the company Dow Corning.

The compositions according to the invention may also comprise a non-emulsifying elastomer.

Non-emulsifying elastomers are described especially in patent applications JP-A-61-194009, EP-A-242219, EP-A-285886 and EP-A-765656, the content of which is incorporated by way of reference.

Spherical non-emulsifying elastomers that may be used include those sold under the names DC 9040, DC 9041, DC 9509, DC 9505 and DC 9506 by the company Dow Corning.

The spherical non-emulsifying silicone elastomer may also be in the form of an elastomeric crosslinked organopolysiloxane powder coated with silicone resin, especially with silsesquioxane resin, as described, for example, in U.S. Pat. No. 5,538,793, the content of which is incorporated by way of reference. Such elastomers are sold under the names KSP-100, KSP-101, KSP-102, KSP-103, KSP-104 and KSP-105 by the company Shin-Etsu.

Other elastomeric crosslinked organopolysiloxanes in the form of spherical powders may be hybrid silicone powders functionalized with fluoroalkyl groups, sold especially under the name KSP-200 by the company Shin-Etsu; hybrid silicone powders functionalized with phenyl groups, sold especially under the name KSP-300 by the company Shin-Etsu.

The content of elastomeric crosslinked organopolysiloxanes in the composition according to the invention may range from 1% to 20% by weight and preferably from 2% to 20% by weight relative to the total weight of the said composition.

Aqueous Phase

The composition according to the invention, in particular when it is in the form of a product cast in the form of a solid emulsion, may comprise an aqueous phase.

The aqueous phase comprises water. A water that is suitable for use in the invention may be a floral water such as cornflower water and/or a mineral water such as Vittel water, Lucas water or La Roche Posay water and/or a thermal water.

The aqueous phase may also comprise organic solvents that are miscible with water (at room temperature—25° C.), for instance monoalcohols containing from 2 to 6 carbon atoms, such as ethanol or isopropanol; polyols especially containing from 2 to 20 carbon atoms, preferably containing from 2 to 10 carbon atoms and preferentially containing from 2 to 6 carbon atoms, such as glycerol, propylene glycol, butylene glycol, pentylene glycol, hexylene glycol, dipropylene glycol or diethylene glycol; glycol ethers (especially containing from 3 to 16 carbon atoms) such as mono-, di- or tripropylene glycol (C₁-C₄)alkyl ethers and mono-, di- or triethylene glycol (C₁-C₄)alkyl ethers, and mixtures thereof.

The aqueous phase may also comprise stabilizers, for example sodium chloride, magnesium dichloride or magnesium sulfate.

The aqueous phase may also comprise any water-soluble or water-dispersible compound that is compatible with an aqueous phase, such as gelling agents, film-forming polymers, thickeners or surfactants, and mixtures thereof.

In particular, a composition of the invention in the form of a cast product of solid emulsion type may comprise an aqueous phase in a content ranging from 5% to 25% and especially from 5% to 20% by weight relative to the total weight of the composition.

According to one preferred mode, the composition according to the invention is in the form of a cast product of anhydrous type.

Pulverulent Phase

The pulverulent phase comprises at least one pigment and optionally at least one filler.

According to one preferred mode, the pulverulent phase contains at least one pigment and at least one filler.

A person skilled in the art will know how to adapt the contents of pulverulent phase, fatty phase and aqueous phase as a function of the form of the composition chosen from an anhydrous cast product and a product cast in the form of a solid emulsion.

Fillers

The term “fillers” should be understood as meaning colourless or white, mineral or synthetic particles of any form, which are insoluble in the medium of the composition irrespective of the temperature at which the composition is manufactured.

The fillers may be mineral or organic, of any form: platelet-shaped, spherical or oblong, irrespective of the crystallographic form (for example lamellar, cubic, hexagonal, orthorhombic, etc). Mention may be made of talc, mica, silica, kaolin, polyamide (Nylon®) powder, poly-β-alanine powder and polyethylene powder, tetrafluoroethylene polymer (Teflon®) powders, lauroyllysine, starch, boron nitride, hollow polymer microspheres such as those of polyvinylidene chloride/acrylonitrile, for instance Expancel® (Nobel Industrie) or of acrylic acid copolymers, silicone resin microbeads (for example Tospearls® from Toshiba), elastomeric polyorganosiloxane particles, precipitated calcium carbonate, magnesium carbonate, magnesium hydrogen carbonate, hydroxyapatite, barium sulfate, aluminium oxides, polyurethane powders, composite fillers, hollow silica microspheres, glass or ceramic microcapsules, and metal soaps derived from organic carboxylic acids containing from 8 to 22 carbon atoms and preferably from 12 to 18 carbon atoms, for example zinc stearate, magnesium stearate, lithium stearate, zinc laurate or magnesium myristate.

According to one preferred mode, use will be made of at least one filler chosen from kaolin, polyamide (Nylon®) powders and acrylic acid copolymers, and mixtures thereof.

The total content of filler(s) in the composition according to the invention may range especially from 0.01% to 40% by weight, preferably from 0.1% to 30% by weight and better still from 1% to 20% by weight relative to the total weight of the said composition.

According to one particularly preferred embodiment, a composition according to the invention comprises from 1% to 10% by weight of fillers, especially from 2% to 8% by weight and in particular from 3% to 5% by weight of fillers relative to the total weight of the said composition.

Pigments

The term “pigments” should be understood as meaning white or coloured, mineral or organic particles of any form, which are insoluble in the physiological medium, and which are intended to colour the composition.

The pigments may be white or coloured, and mineral and/or organic. Among the mineral pigments, mention may be made of titanium dioxide, optionally surface-treated, zirconium oxide or cerium oxide, and also zinc oxide, iron oxide (black, yellow or red) or chromium oxide, manganese violet, ultramarine blue, chromium hydrate and ferric blue, and metal powders such as aluminium powder or copper powder.

Among the organic pigments, mention may be made of carbon black, pigments of D&C type and lakes, especially lakes based on cochineal carmine or on barium, strontium, calcium or aluminium.

In the case of a cast product, when it is a makeup product, a pigment content ranging from 1% to 50% by weight, preferably from 5% to 40% by weight and in particular from 5% to 30% by weight relative to the total weight of the said composition will generally be used.

As stated previously, a composition according to the invention advantageously comprises a pigment content of greater than or equal to 5% by weight relative to the total weight of the said composition, especially greater than or equal to 10% by weight, in particular greater than or equal to 15% by weight and more particularly ranging from 15% to 30% by weight relative to the total weight of the said composition.

The composition according to the invention may comprise an additional pulverulent dyestuff, other than the pigments described previously, which may be chosen especially from nacres and other interference pigments, and glitter flakes, and mixtures thereof.

The nacres may be chosen from white nacres such as mica coated with titanium or with bismuth oxychloride, coloured nacres such as titanium mica coated with iron oxides, titanium mica coated especially with ferric blue or with chromium oxide, titanium mica coated with an organic pigment of the abovementioned type, and also nacres based on bismuth oxychloride.

The nacres may be present in the composition in a content ranging from 0.1% to 50% by weight, preferably ranging from 0.1% to 40% by weight and preferentially ranging from 0.1% to 30% by weight relative to the total weight of the composition.

Other Ingredients

The composition may comprise other ingredients (adjuvants) usually used in cosmetics, such as preserving agents, cosmetic active agents, moisturizers, water-soluble or liposoluble dyes, UV-screening agents, thickeners, water, surfactants and fragrances.

The liposoluble dyes are, for example, Sudan red, DC Red 17, DC Green 6, β-carotene, soybean oil, Sudan brown, DC Yellow 11, DC Violet 2, DC Orange 5 and quinoline yellow. The water-soluble dyes are, for example, beetroot juice and caramel.

Needless to say, a person skilled in the art will take care to select the optional adjuvants(s) added to the composition according to the invention, such that the advantageous properties intrinsically associated with the composition in accordance with the invention are not, or are not substantially, adversely affected by the envisaged addition.

The composition in the form of a cast product may be prepared by mixing the ingredients of the pulverulent phase, of the fatty phase and, where appropriate, of the aqueous phase for the cast products of solid emulsion type, with heating, and then leaving the mixture to cool in a mould.

The invention is illustrated in the examples presented below as non-limiting illustrations of the field of the invention.

Unless otherwise indicated, the values in the examples below are expressed as weight percentages relative to the total weight of the composition.

### EXAMPLE 1

### Coloured Foundation Composition

| Ingredients/commercial references | Weight % |
|---|---|
| Red iron oxide | 15 |
| (CI: 77491) | |
| Cyclopentadimethylsiloxane | 47.07 |
| (Dow Corning 245 Fluid) | |
| Trimethyl siloxysilicate resin | 6 |
| (SR 1000 ® from Mementive Performance Materials) | |
| Block copolymer [polydimethylsiloxane (dp = 100)/ | 2.57 |
| polyamide] (Dow Corning 2-8179 ® Gellant | |
| from Dow Corning) | |
| Nylon-12 microspheres (particle size: 5 microns) | 3.6 |
| Ethylene homopolymer | 4 |
| (Performalene 400 Polyethylene ® from New | |
| Phase Technologies) | |
| Mixture of linear long-chain (C₃₀-C₅₀) fatty alcohol and of | 1 |
| hydrocarbon of the same carbon number (80/20) | |
| (Performacol 550-L Alcohol ® from New | |
| Phase Technologies) | |
| Isododecane | qs 100 |

Procedure

The non-particulate constituents were weighed out in an oil-circulated jacketed heating pan heated to 95-100° C.

After homogenization, the pigments dispersed in the medium and then the talc were added.

The mixture was then poured into dishes.

### EXAMPLE 2

### Hot-Cast Foundation

| Ingredients/commercial references | Weight % |
|---|---|
| Kaolin | 4.90 |
| Titanium dioxide | 20 |
| Red iron oxide | 6.5 |
| (CI: 77491) | |
| Copernicia cerifera (carnauba) wax | 2.5 |
| (Carnauba Wax SP 63 from the company Strahl & Pitsch) | |
| Microcrystalline wax (C20-C60) | 5 |
| (Microwax HW from the company Paramelt) | |
| Phenyl trimethicone | 5 |
| (Dow Corning 556 Cosmetic Grade Fluid from Dow Corning) | |
| Mixture of crosslinked polydimethylsiloxane and of | 14.15 |
| polydimethylsiloxane (6 cSt) (24/76) | |
| (KSG 16 from Shin-Etsu) | |
| Isononyl isononanoate | qs 100 |
| (Wickenol 151 from the company Alzo) | |

This composition is prepared according to the procedure described in Example 1, the kaolin being introduced at the end of the preparation protocol.

It was also characterized according to the protocol described previously for characterization by DSC, as having a melting point of 66° C. in DSC with a released energy of 0.15 W/g. Everything melted at 95° C.

This composition was evaluated by a panel of 23 women from 20 to 40 years old, having normal to combination skin. The uniformity of the makeup result after application with an applicator as defined in FIGS. 1 to 3 is evaluated in comparison with application with a sponge.

Protocol

The handling and application protocol is as follows:
the sponge is passed three times over the composition;
the applicator according to the invention is passed three times over the composition;
application to the face of the women is performed by a beautician, on one half of the face with the sponge and on the other half with the rotary applicator;
an expert grades the visibility of the “makeup marks” from photos taken of the made-up models, according to the protocol described previously:

grade 1=no visible marks of irregular makeup (uniform application)
grade 2=visible marks of irregular makeup (non-uniform application).

Results

The product applied with the applicator according to the invention gives “no visible marks” for 70% of the women.

The product applied with the sponge gives “no visible marks” for 9% of the women.

Far less irregular makeup is thus statistically obtained for the product applied with a rotary applicator according to the invention, compared with the same product applied with a sponge.

### EXAMPLE 3

### Compact Complexion Cream

| Ingredients/commercial references | Weight % |
|---|---|
| Anatase titanium oxide coated with aluminium stearoyl | 14.72 |
| glutamate (NAI-TAO-77891 from Miyoshi Kasei) | |
| Barium sulfate coated with n-lauroyllysine (95/5) | 7.40 |
| (CI: 77120) (LLD-5 BASO4 (PL) from Daito Kasei Kogyo) | |
| Red iron oxide coated with aluminium stearoyl glutamate | 0.75 |
| (NAI-C33-8001-10 from Miyoshi Kasei) | |
| Black iron oxide coated with aluminium stearoyl glutamate | 0.41 |
| (NAI-C33-7001-10 from Miyoshi Kasei) | |
| Yellow iron oxide coated with aluminium stearoyl glutamate | 3.12 |
| (NAI-C33-9001-10 from Miyoshi Kasei) | |
| Isononyl isononanoate | 21 |
| Nylon-12 | 8 |
| Polyethylene | 1.60 |
| (Performalene 500 L Polyethylene ® from New | |
| Phase Technologies) | |
| Hydrogenated jojoba oil | 6.0 |
| (Jojoba Wax Flakes from Desert Whale) | |
| Polydimethylsiloxane 5 cSt | qs 100 |
| (Dow Corning 200 Fluid ® from Dow Corning) | |

This composition is prepared according to the procedure described in Example 1, the Nylon® being introduced at the end of the preparation protocol.

It has a melting point of 56° C. by DSC, with a released energy of 0.3 W/g. The melting peak is narrow. Everything melted at 90° C.

The application of this composition with an application device equipped with a rotary application member as described in FIGS. 1 to 3 is evaluated on a panel of 10 women from 25 to 50 years old, using a foundation, and having combination skin.

The application area is the face.

The application, performed once, is free.

The application, the perceptions and the makeup result are evaluated.

The application device allows good handling and is very manipulable: the application is precise on all the areas of the face, including the contour of the eyes and the sides of the nose. In addition, it has the advantage of not soiling the fingers with the foundation.

Finally, the users appreciate the light massaging afforded by stroking on application of the said composition with this device.

In terms of makeup result, the applied film is very thin, uniform and free of marks. The makeup is discreet and natural.

1. A cosmetic assembly for making up and/or caring for keratin materials, the cosmetic assembly comprising:
a container containing at least one solid cosmetic composition, the cosmetic composition comprising at least one liquid fatty phase, at least one wax, and at least 5% by weight of pigments relative to a total weight of the cosmetic composition, and having a melting point of greater than 30° C.; and
an application device for applying the cosmetic composition, wherein the application device comprises an application member comprising an application surface that is capable, in response to its movement in engagement with the keratin materials, of rotating about at least one axis or centre of rotation.

2. The assembly according to claim 1, wherein the cosmetic composition has a hardness of less than or equal to 350 g.

3. The assembly according to claim 1, wherein the cosmetic composition is an anhydrous cast product.

4. The assembly according to claim 1, wherein the cosmetic composition is a cast product of solid emulsion type.

5. The assembly according to claim 1, wherein the cosmetic composition comprises from 5% to 50% by weight of an ester oil.

6. The assembly according to claim 5, wherein the ester oil is chosen from isodecyl neopentanoate, isocetyl octanoate, isononyl isononanoate, isodecyl isononanoate, tridecyl isononanoate, hexyl laurate, 2-hexyldecyl laurate, isopropyl myristate, isocetyl myristate, isotridecyl myristate, 2-octyldodecyl myristate, isopropyl palmitate, 2-ethylhexyl palmitate, isooctyl palmitate, isocetyl palmitate, isodecyl palmitate, isostearyl palmitate, 2-octyldecyl palmitate, isopropyl isostearate, 2-octyl-dodecyl stearate, isostearyl isostearate; 2-octyldodecyl erucate, and mixtures thereof.

7. The assembly according to claim 1, wherein the cosmetic composition comprises at least two waxes having differing polarities.

8. The assembly according to claim 1, wherein the cosmetic composition comprises at least one apolar wax with a melting point ranging from 50 to 100° C. and at least one polar wax with a melting point ranging from 40 to 100° C.

9. The assembly according to claim 1, wherein the cosmetic composition comprises at least one hydrocarbon-based polar wax chosen from beeswax, rice bran wax, carnauba wax, hydrogenated jojoba oil, and an alcohol wax, and at least one hydrocarbon-based apolar wax chosen from a polyethylene wax and a microcrystalline wax, and mixtures thereof.

10. The assembly according to claim 1, wherein the cosmetic composition comprises at least one carnauba wax and at least one microcrystalline wax.

11. The assembly according to claim 1, wherein the cosmetic composition comprises at least one silicone resin in a proportion of from 0.1% to 40% by weight relative to its total weight.

12. The assembly according to claim 1, wherein the cosmetic composition comprises at least from 0.01% to 40% by weight material relative to its total weight.

13. The assembly according to claim 1, wherein the application device is a roll.

14. The assembly according to the claim 13, wherein the roll has an application surface of foam with open or closed cells, flocking, elastomer, sintered material, woven material, or nonwoven material.

15. The assembly according to claim 1, wherein the cosmetic composition is chosen from a makeup rouge, an eyeshadow, a face powder, a foundation, a concealer product, a lipstick, a body makeup product, a facial care product, a body care product, or an antisun product.

16. A process for making up and/or caring for a keratin material, the process comprising:
loading an amount of a solid composition using an application member comprising an application surface that is capable, in response to its movement in engagement with keratin materials, of rotating about an axis or a centre of rotation; and
applying the loaded composition using the application member onto the keratin material,
wherein the composition comprises at least one liquid fatty phase, at least one wax and at least 5% by weight of pigments relative to the total weight of the said composition, and has a melting point of greater than 30° C.

17. The process according to claim 16, wherein the composition has a hardness of less than or equal to 350 g.

18. The process according to claim 16, wherein the composition comprises at least from 0.01% to 40% by weight of filler material relative to its total weight.

19. The process according to claim 16, wherein the composition is chosen from a makeup rouge, an eyeshadow, a face powder, a foundation, a concealer product, a lipstick, a body makeup product, a facial care product, a body care product, or an antisun product.

20. The assembly according to claim 1, wherein the cosmetic composition comprises from 5% to 50% by weight of a polar oil.

21. The assembly according to claim 1, wherein the cosmetic composition comprises at least one apolar wax and at least one polar wax.

22. The assembly according to claim 1, wherein the cosmetic composition comprises at least one silicone resin.
